(19) United States
(12) Patent Application Publication
(10) Pub. No.: US 2016/0066804 A1
(43) Pub. Date: Mar. 10, 2016

(54) MEASUREMENT ELECTRODE ASSEMBLY

(71) Applicant: TMS INTERNATIONAL B.V., Ej Oldenzaal (NL)

(72) Inventor: Jan Henrik Johan Peuscher, E J Oldenzaal (NL)

(21) Appl. No.: 14/782,696

(22) PCT Filed: Mar. 27, 2014
(86) PCT No.: PCT/EP2014/056160

§ 371 Date: Oct. 6, 2015

(30) Foreign Application Priority Data
Apr. 10, 2013 (DE) 102013006276.4
Oct. 29, 2013 (DE) 102013018478.9

(51) Int. Cl. A61B 5/04; A61B 5/00

(52) U.S. Cl. CPC A61B 5/04; A61B 5/6803

(57) ABSTRACT

The invention relates to a measurement electrode assembly for removable fastening to a flexible carrier material in the form of a belt, a hood or similar means having a mating side and a connection side, which has an electrical contacting element, wherein an absorbent insert, in particular a felt insert, is designed protruding from the mating surface, and a fastening part which fastens the absorbent insert. According to the invention, the fastening part consists of a sintered silver-chloride material or receives such a material, wherein the absorbent insert is interchangeably screwed, pressed, latched, pushed and/or wedged into the fastening part. There is furthermore a mechanical stabilization device on the mating side, enclosing the absorbent insert, which has the design of a collar and/or appendage resembling a spider leg.

The invention relates to a measurement electrode arrangement for detachably fastening to a flexible carrier material in the form of a band, a head cap or similar means having a contact side and a connection side, which connection side is provided with an electrical contacting element, wherein projecting from the contact side is formed an absorbent insert, in particular a felt insert, as well as a fastening part that secures the absorbent insert, according to claim 1.

An electrode for electromedical devices is known from DE 1 965 195. This prior art starts from electrodes which normally exhibit a small-sized electrode sponge filled with an electrically conductive liquid, with the electrode sponge resting upon the skin and providing for a uniform current transition from the electrode to the skin. Such electrodes are fixed to the test person either via suction cups or are held by means of tapes even with the use of hook and loop fasteners.

Electrode sponges or felt-like electrode inserts can be disinfected or formed as disposable items. The electrode according to DE 1 965 195 starts from a pliable film which exhibits, on one side, an electrically conducting surface which is associated with a means to connect the electrode cable. This known device is moreover characterized by a pad applied to the electrically conducting surface of the film, which pad is of an electrically poorly conducting or insulating material as a carrier for an electrically conducting adhesion means of preferably pasty consistency. The pad serves the purpose of creating a defined contact resistance between film and skin and to thus obtain a uniform current transition between film and skin across the entire electrode surface. The pad may be of cotton, linen or synthetic fabric or of fiber paper, wherein the pad is fixed on the conductive film in a material-bonded manner such that the material bond is only in the form of a grid, in rings or points while exposing the preponderant part of the film surface. In order to create sufficient electrical contact with the test person's skin, however, the presented known electrode needs to exhibit a minimum surface area and to abut across this entire surface area, i.e. at a defined contact pressure. However, the more extensive the contact surface is realized, the more problematic the adjusting of reproducible mechanical forces and the thus resulting electrical conditions are.

In the self-adhesive sensor element according to DE 10 2007 024 238 A1, the sensor system is intended to be applied to the patient in a possibly discreet manner and/or to be integrated in textile or flex material. The teaching therein intends to do without additional, permanent contact pressure. At the sensor element moreover at least one sensor, an electronic element, an actuator and/or a further element are present. The surface is equipped with elastically deformable nap-shaped projections which return to their initial position or initial form after the self-adhesive connection is released from the respective adhesive body.

The surface of the nap-shaped projections is formed to be spherically curved at least in the region which is brought into touch contact with the respective adhesive body. This curvature preferably can be formed to be spherical but also to follow an ellipse or another convex or concave shapes.

In order to ensure adhesion, the nap-shaped projections are provided with specific coupling and functional properties. The nap-shaped projections can be formed directly on the carrier layer or preferably attached thereto in a non-detachable manner. The spherically curved nap-shaped projections generate suction processes while being in contact with the surface of adhesive bodies which cause a vacuum to be formed between the adhesive body and the surface of nap-shaped projections upon deformation of the nap-shaped projections. So as to achieve the discussed self-adhesive properties, the prior sensor element needs the mentioned projections to be of a large-area configuration. Furthermore, there exist problems with respect to the reproducibility of mechanical force effects between that part of the projections that is effective as the electrodes and the test person's skin surface. This is because the deformations required to generate suction forces naturally also result in an altered contact surface having in turn an altered contact transition resistance.

Form the afore-mentioned it is therefore a task of the invention to propose a further developed measurement electrode arrangement for detachably fastening to a flexible carrier material in the form of a band, a head cap or similar means, which can be produced at low cost, be realized in miniaturized form, and which under all circumstances ensures reproducible electrical properties, in particular contact transition resistances to a test person's skin surface.

The task of the invention is solved by a measurement electrode arrangement according to the feature combination as per claim 1, wherein the subclaims at the least comprise advantageous configurations and further developments.

Accordingly, the basis herein is a measurement electrode arrangement for detachably fastening to a flexible carrier material in the form of a band, a head cap or similar means, wherein the electrode arrangement has a contact side and an electrical connection side. The electrical connection side is provided with an electrical contacting element. An absorbent insert, in particular a felt insert is formed to be projecting from the contact side. Moreover, a fastening part is present that secures the absorbent insert.

The absorbent insert may be of a cylindrical or pin-shaped felt material, for example, so as to realize a point contact electrode. The side or the respective end of the felt insert getting in contact with the test person's skin surface may be formed spherically or semi-spherically or else conical or truncated conical.

According to a first embodiment, the fastening part itself is either formed of a conducting material or receives such a material. Optional embodiments of the conducting material are carbon-containing plastics, silver-containing textile materials, noble metals and bodies of sintered silver chloride.

The metal of the electrode together with the contact gel and the skin forms a galvanic cell whose voltage may be several hundreds of millivolts and differs between individual electrodes. The discharging cycle of these galvanic cells may be from 10 to 20 minutes and is removed via filters that eliminate low frequencies (<0.2 Hz) from the measuring signal. Furthermore, the discharging process is not uniform but takes place in surges, which likewise impedes the filtering. The exemplary use of silver chloride minimizes the voltage of the galvanic cells and reduces the cascade-like discharging behavior.

The absorbent insert is exchangeably introduced into the fastening part, in particular screwed, pushed, snapped, pressed and/or clamped into the fastening part.

Furthermore, on the contact side, a mechanical stabilization device which surrounds the absorbent insert and the fastening part, respectively, is provided according to the invention. This mechanical stabilization device for instance has the form of a sleeve and/or spider leg-like projections. The sleeve and the spider leg-like projections may be made of an elastic or elastomer material, for example. The stabilizing prevents the absorbent material from sliding across the contacted skin and/or the size of the contact surface between the electrode and skin and thus the impedance (consequently the amplitude of the measuring signal) from changing during measurement. Furthermore, a compensation of variations in the contact pressure (change of the impedance) is given, and the sheathing prevents the absorbent insert from rapidly drying out.

When the respective electrode is contacted with the surface of a test person's skin, the mechanical stabilization device provides for a defined position of the electrodes that are preferably realized in a miniaturized form. At the same time, a tightening of the skin surface and thereby an improved contacting is performed.

The electrical contacting element is in electrical connection to the conductive electrode body, this material in turn being electrically connected to the absorbent insert. For the purpose of deploying the relevant electrical functions, the absorbent insert is impregnated with a conductive liquid and/or provided with a corresponding conductive gel.

In the event that the absorbent insert is impregnated with a conductive liquid, the configuration of the electrode housing will ensure that the absorbent inserts of two electrodes are not electrically short-circuited through the carrier material (e.g. band, cap) that is potentially likewise impregnated by the liquid. In addition, a rapidly drying textile may be used for the carrier material.

It should be noted here that the term “absorbent insert” also implies an insert which itself is conductive, e.g. by being made of conductive microparticles or containing such particles. The absorbent insert unifies here the desired properties of resilience and conductivity as well as exchangeability, the latter for hygienic reasons.

The screwing, clamping or other fastening process with respect to the absorbent insert in the fastening part allows the absorbent insert to be removed after use and to be replaced with a new, hygienically perfect insert.

In one embodiment, the fastening means is formed as a hollow body which has a sintered insert in its interior, wherein the absorbent insert is screwed, pushed, snapped, pressed and/or clamped into a passage opening of the sintered insert.

To this end, the passage opening may have a thread for screwing in the cylindrically or pin-like formed part of the absorbent insert.

The absorbent insert extends up to the contact side and penetrates the fastening part. Furthermore, a force is applied on the absorbent insert which forms a vector toward the contact side, thus minimizing the contact transition resistance to the test person's skin surface.

The hollow body as the fastening part is made of an electrically insulating material.

The fastening part preferably is of a multi-piece configuration so as to fix the same to the band or the head cap via an opening that is present there. Preferably, a push button-like fixing of the fastening part to the band or head cap is used.

The sleeve and/or the spider leg-like projections as the stabilizing part surround the free end of the absorbent insert at a radial spacing.

The spider leg-like projections provided in one embodiment exhibit a radial orientation starting from the absorbent insert and have a bending or spherical extension.

The bending or spherical extension is realized such that, when the band or head cap is applied to the test person, the projections will yield and laterally move away from the center of the absorbent insert. Hereby, a tightening of the skin surface takes place and the electrical contacting is optimized. It should be noted in this place that the mechanical stabilization device has no electrical function and therefore is made of a non-conductive material or is formed to be isolated relative to the absorbent insert or other conductive elements.

The sleeve and/or the spider leg-like projections of the stabilization device are in connection with the fastening part. This connection may be obtained or realized concurrently with the push button-like fixing of the fastening part to the band or head cap.

In a further inventive embodiment, a free end of the absorbent insert projecting out of the fastening part on the contact side may be covered with an elastic cap or similar means. The cap is thereby fixed between the fastening part and the respective end of the absorbent insert, whereby an additional force vector of the absorbent insert toward the test person's skin surface can be achieved. The cap may have a defined amount of conductive liquid in its interior which is released when the cap is put on and fixed to the fastening part, to come then in contact with and impregnate the absorbent insert.

In a second embodiment of the invention, the fastening part is an insulating material and has a pot shape. The absorbent insert is in this embodiment exchangeably screwed, pushed, snapped, pressed and/or clamped into the interior of the fastening part. In this embodiment as well, a mechanical stabilization device is present on the contact side which in particular surrounds the fastening part and is fixed to the same in a form-fitting and/or force-transmitting manner.

In this case, the mechanical stabilization device protrudes beyond the outer edge of the fastening part, yet is at least approximately flush with the absorbent insert or the tip of the absorbent insert. The respective sleeve is connected with the fastening part in a form-fitting and/or force-transmitting exchangeable manner, i.e. can be easily replaced.

The sleeve is realized as a flexible ring having concentric protrusions and setbacks, wherein the concentric protrusions and setbacks serve the purpose of fixing the position of and correspondingly stabilizing the electrode arrangement as already mentioned above. Grid inaccuracies are thereby prevented.

In this embodiment, the contacting element is in direct connection with the absorbent insert and, for example, is guided through a lateral opening in the fastening part into the interior thereof and can get into contact with the absorbent insert there.

The fastening part has an opening on the bottom side for introducing a conductive liquid which reaches the absorbent insert via this opening.

The absorbent insert is formed to be correspondingly complementary to the pot shape of the fastening part and can thus be introduced into the open side of the pot. The insert may comprise a truncated cone-shaped or conical tip, yet may also be configured spherically or semi-spherically, in order to establish, as far as possible, a point contact with the test person's skin surface.

The invention will be explained below in greater detail using figures and exemplary embodiments.

Hereby shown are in:

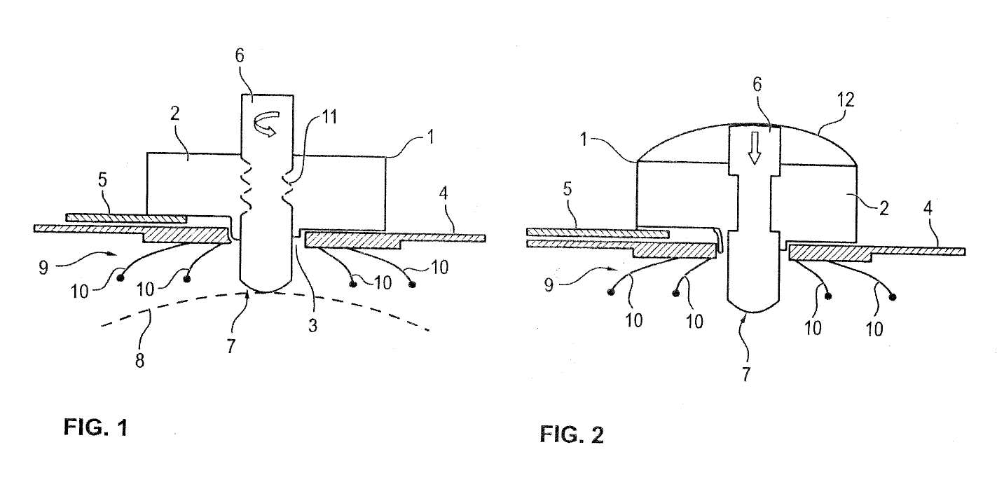

FIG. 1 a longitudinal sectional view of the inventive measurement electrode arrangement according to a first exemplary embodiment;

FIG. 2 a representation in the form of a longitudinal cut of the measurement electrode arrangement according to a second exemplary embodiment; and

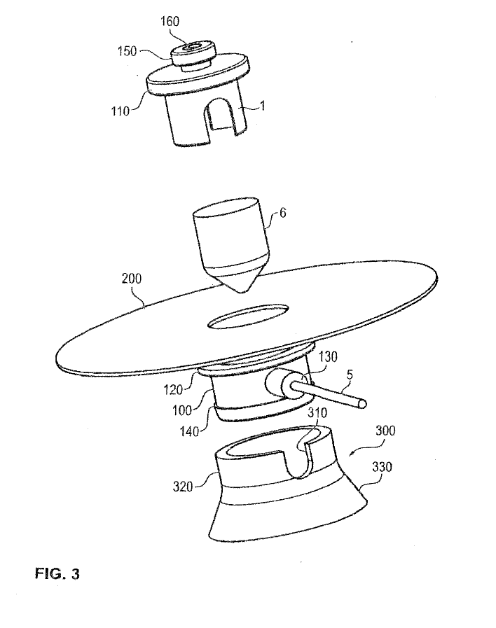

FIG. 3 a perspective representation of a third embodiment in an exploded view.

The measurement electrode arrangement according to the Figures is firstly based on a fastening part 1. This part can be formed as a two-piece hollow body and comprise sintered silver chloride 2 in its interior.

The fastening part 1 can be fixed in a push button-like manner (reference numeral 3) to a fabric tape 4 in a recess present therein. A contacting element, e.g. in the form of an electrode wire 5, extends up to the interior of the fastening part 1 and the sintered silver chloride material present therein.

The fastening part 1 is penetrated by an absorbent insert 6.

The absorbent insert 6 is also in communication with the sintered silver chloride material 2. On the contact side, the absorbent insert exits or protrudes and comprises a spherical shape 7 at its end pointing toward a test person's skin. The skin surface is symbolically depicted in dashed lines by reference numeral 8, wherein in the representation according to FIG. 1, the complete establishment of contact of the fabric tape 4 and thus the measurement electrode arrangement has not been implemented yet.

A mechanical stabilization device 9 surrounds the absorbent insert 6 at its spherically formed part 7.

The mechanical stabilization device 9 exhibits, for instance, a plurality of spider leg-like projections 10 or is formed as a sleeve (not shown).

As can be seen from the sectional representations as per FIGS. 1 and 2, the fastening means 1 is realized as a hollow body, wherein the absorbent insert 6 is screwed according to FIG. 1 or snapped or clamped according to FIG. 2 into a corresponding passage opening.

The screw thread is outlined in broken lines in FIG. 1 by reference numeral 11.

It is apparent from the Figures that the absorbent insert 6 extends up to the contact side and penetrates the fastening part 1. The thread as per FIG. 1 allows a force to be applied which forms a vector toward the contact side.

It is likewise possible in the exemplary embodiment as per FIG. 2 for a force vector toward the contact side to be generated in that an elastic tape or an elastic cap 12 presses the absorbent insert 6 in the arrow direction as shown, for example.

The spider leg-like projections 10 surround the free end of the absorbent insert 6, i.e. the end directed toward the test person's skin surface 8 is radially spaced. The individual projections 10 have a curve or spherical extension as is symbolically visible in FIGS. 1 and 2.

The absorbent insert 6 can be easily withdrawn from the fastening part and replaced with a new insert so as to meet all hygienic requirements.

According to the perspective representation as per FIG. 3 which shows a third embodiment in an exploded view, the measurement electrode arrangement is constituted by a fastening part 1. Here, it may be a plastic injection molded part.

The fastening part 1 of this embodiment is realized in a pot shape and has an inner hollow space into which the absorbent insert 6 can be pushed and/or snapped or clamped.

The fastening part according to the embodiment as per FIG. 3 is constituted by two elements, namely part 1 and part 100.

The outer dimensions of part 1 in the area of the cylindrical portion are adapted to the inner diameter of the lower part 100. Consequently, part 1 (upper part) including the inserted insert can be inserted into and connected to part 100 (lower part) in a push button-like manner. At the same time, a textile material 200 is retained clamped between the upper part's 1 circumferential collar 110 and a circumferential collar 120 of the lower part 100. The textile material 200 may be a portion of a tape of a head cap or similar means for fixing the electrode arrangement to a test person's body part.

A lateral protrusion 130 of the lower part 100 has a passage opening for connecting an electrical contacting element 5. A recess 310 corresponding to the outer dimensions of part 130 is formed in a sleeve 300 so that a ring portion 320 of sleeve 300 can be put over the lower part 100. A spreading protrusion 140 on the lower part 100 securely fixes the sleeve 300, wherein a corresponding annular groove (not shown in FIG. 3) may further be present in the sleeve 300.

The sleeve 300 opens, e.g. in a conical shape, toward the side that is open downwards in the mounting state. In this conical portion 330 of the sleeve 300, lower side concentric protrusions and setbacks may be provided for fixing the position of the entire arrangement with respect to a test person's skin surface,

The sleeve 300 may be manufactured of an elastomer, in particular rubber-like material.

In the upper part 1, a projection 150 having a passage opening 160 is further situated. The absorbent insert 6 may be impregnated with conductive liquid via this passage opening 160.

1. Measurement electrode arrangement for detachably fastening to a flexible carrier material (4) in the form of a band, a head cap or similar means having a contact side and a connection side, which connection side is provided with an electrical contacting element (5), wherein projecting from the contact side is formed an absorbent insert (6), in particular a felt insert, as well as a fastening part (1) that secures the absorbent insert (6),
characterized in that
the fastening part (1) is realized in a pot shape, wherein the absorbent insert (6) is exchangeably screwed, pushed, snapped, pressed and/or clamped into the interior of the fastening part (1), and furthermore, on the contact side, a mechanical stabilization device is provided so as to surround either the absorbent insert directly or the fastening part (100), is made of sintered silver chloride (2) or receives such a material, wherein the mechanical stabilization device has the shape of a sleeve (300) and/or spider leg-like projections (10) and is connected with the fastening part (100) in a form-fitting and/or force-transmitting exchangeable manner.

2. Arrangement according to claim 1,
characterized in that
the fastening part (1) is formed as a sintered insert of silver chloride.

3. Arrangement according to claim 1,
characterized in that
the contacting element (5) is in electrical connection to the absorbent insert (6) and/or the sintered insert (2).

4. Arrangement according to claim 1,
characterized in that
the pot-shaped fastening part (1) comprises a passage opening for introducing a cylindrical or pin-shaped part of the absorbent insert (6).

5. Arrangement according to claim 1,
characterized in that
the absorbent insert (6) extends up to the connection side and penetrates the fastening part (1) and is applied by a force which forms a vector toward the contact side.

6. Arrangement according to claim 1,
characterized in that
the fastening part comprises a bottom side (110) having an opening (160) for introducing a conductive liquid.

7. Arrangement according to claim 1,
characterized in that
the fastening part (1) is of a multi-piece configuration so as to fix the same to the band (4) or the head cap via an opening that is present there.

8. Arrangement according to claim 7,
characterized by
a push button-like fixing of the fastening part to the band (4) or head cap.

9. Arrangement according to claim 1,
characterized in that
the sleeve and/or the spider leg-like projections (10) surround the free end of the absorbent insert (6) on the contact side at a radial spacing.

10. Arrangement according to claim 1,
characterized in that
starting from the absorbent insert (6), the spider leg-like projections (10) are radially oriented and exhibit a curvature or spherical extension.

11. Arrangement according to claim 1,
characterized in that
the sleeve (300) is formed as a flexible ring having concentric protrusions and setbacks for position fixing.

12. Arrangement according to claim 1,
characterized in that
the absorbent insert (6) is impregnated with conductive liquid and/or comprises a conductive gel layer.

13. Arrangement according to claim 1,
characterized in that
the absorbent insert (6) is formed to be correspondingly complementary to the pot shape of the fastening part (1) and comprises a protruding, spherical, semi-spherical, truncated cone-shaped or conical tip which can be brought into contact with a test person's skin surface.
